(19) United States
(12) Patent Application Publication
(10) Pub. No.: US 2014/0107658 A1
(43) Pub. Date: Apr. 17, 2014

(54) Surgical Instrument

(71) Applicants: Michael J. Yaremchuk, Lynnfield, MA (US); Eric Lui, West Chester, PA (US); Jay Schlink, West Chester, PA (US)

(72) Inventors: Michael J. Yaremchuk, Lynnfield, MA (US); Eric Lui, West Chester, PA (US); Jay Schlink, West Chester, PA (US)

(21) Appl. No.: 13/649,428

(22) Filed: Oct. 11, 2012

(51) Int. Cl. A61B 17/88

(52) U.S. Cl. 606/99

(57) ABSTRACT

A surgical instrument is described that is configured to apply a force to first and second surfaces to secure the first surface relative to the second surface during fixation of the first and second surfaces.

### TECHNICAL FIELD

The present disclosure relates to a surgical instrument configured to hold an implant in a desired position against an underlying honey structure, so the implant can be secured to the honey structure, while also providing compression between the implant and the underlying honey structure such that gaps between implant and the underlying honey structure are minimized

### BACKGROUND

Implants are often used in cosmetic and reconstructive surgical procedures. If a honey structure of a patient, for example a mandible or chin, is deformed or damaged an implant can be positioned against the honey structure and secured to the honey structure in a desired position such that the implant creates a natural or otherwise desired appearance. During a surgical implantation procedure an incision can be made in the skin exposing the damaged or deformed area of the honey structure such that the implant can be placed next to the honey structure in the desired position. The implant is then held in place while fasteners, such as bone screws, are used to secure the implant relative to the honey structure in the desired position.

Typically the implant is held in the desired position by hand, for instance by the surgeon placing a finger against the implant and applying a force in a direction toward the honey structure. This can lead to a non-uniform fit between the implant and the honey structure such that gaps exist between the implant and the honey structure. Gaps or other non-uniform fits between the implant and honey structure are undesirable as they can lead to an uneven or an unnatural appearance.

A surgical instrument that is configured to hold an implant against a honey structure in a desired position while the implant is secured to the honey structure and also provide a compressive force to the implant and the honey structure such that gaps or other non-uniform fits are minimized could improve the outcome of cosmetic and reconstructive surgical procedures.

### SUMMARY

In one embodiment the present disclosure relates to a surgical instrument configured to secure an implant relative to a mandible. The surgical instrument can include a first arm and a second arm that are pivotally connected at a pivot point. The first arm can include a first proximal portion, a first distal portion, and a first body portion that extends from the first proximal portion to the first distal portion. The first distal portion can further include a grip member that is configured to grip the mandible. The second arm can include a second proximal portion, a second distal portion, and a second body portion that extends from the second proximal portion to the second distal portion. The second distal portion can carry an implant retention member that s configured to abut the implant at a first location of the second arm. The second distal portion further carrying a piercing portion that is configured to pierce the implant, the piercing portion disposed at a second location of the second arm that is spaced from the first location of the second arm.

### BRIEF DESCRIPTION OF THE DRAWINGS

The foregoing summary, as well as the following detailed description of the preferred embodiments of the application, will be better understood when read in conjunction with the appended drawings. For the purposes of illustrating various embodiments, drawings are shown. It should be understood, however, that the present disclosure is not limited to the precise arrangement, structures, features, embodiments, aspects, and instrumentalities shown, and that the arrangements, structures, features, embodiments, aspects and instrumentalities shown may be used singularly or in combination with other arrangements, structures, features, aspects, embodiments and instrumentalities. In the drawings:

FIG. 1A is a perspective view of a portion of a skull including a mandible, and an implant configured to be attached to the mandible;

FIG. 1B is a perspective view of the mandible and the implant illustrated in FIG. 1A, the implant positioned against the mandible;

FIG. 2A is a side elevation view of a surgical instrument engaging the implant and the mandible illustrated in FIG. 1A;

FIG. 2B is a perspective view of the surgical instrument, implant, and mandible illustrated in FIG. 2A;

FIG. 2C is another perspective view of the surgical instrument, implant, and mandible illustrated in FIG. 2A;

FIG. 3A is a perspective view of the implant illustrated in FIG. 2A in an open configuration;

FIG. 3B is another perspective view of the implant illustrated in FIG. 3A in the open configuration;

FIG. 3C is a front elevation view of the implant illustrated in FIG. 3A in the open configuration;

FIG. 3D is a side elevation view of the implant illustrated in FIG. 3A in the open configuration;

FIG. 4A is a perspective view of the implant illustrated in FIG. 3A in a closed configuration;

FIG. 4B is another perspective view of the implant illustrated in FIG. 4A in the closed configuration;

FIG. 4C is a front elevation view of the implant illustrated in FIG. 4A in the closed configuration;

FIG. 4D is a side elevation view of the implant illustrated in FIG. 4A in the closed configuration.

### DETAILED DESCRIPTION OF ILLUSTRATIVE EMBODIMENTS

Certain terminology is used in the following description for convenience only and is not limiting. The words “right”, “left”, “lower”, “upper”, “bottom”, and “top” designate directions in the drawings to which reference is made. The words “inwardly” and “outwardly” refer to directions toward and away from, respectively, the geometric center of the bone fixation element, instruments and designated parts thereof. The words, “anterior”, “posterior”, “superior”, “inferior”, “medial”, “lateral” and related words and/or phrases designate preferred positions and orientations in the human body to which reference is made and are not meant to be limiting. The terminology includes the above-listed words, derivatives thereof and words of similar import.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. Further, reference to values stated in ranges includes each and every value within that range. All ranges are inclusive and combinable. Certain features of the invention which are described herein in the context of separate embodiments may also be provided in combination in a single embodiment. Conversely, various features of the invention that are described in the context of a single embodiment may also be provided separately or in any subcombination.

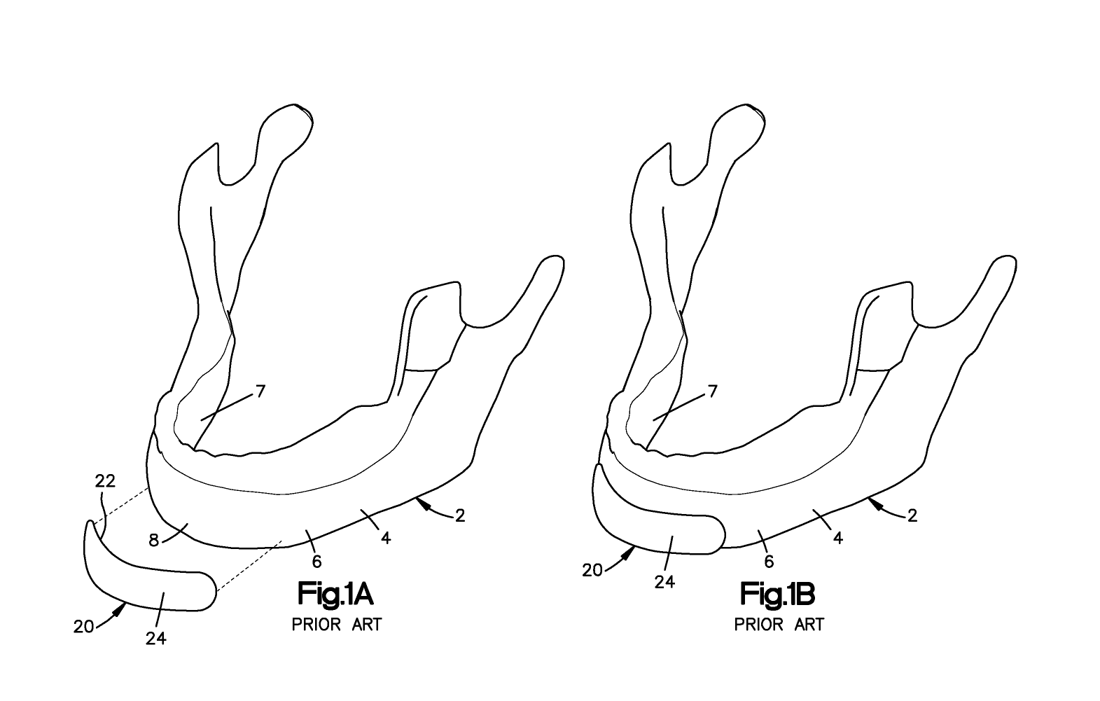

Referring to FIGS. 1A and 1B, a skull portion 2 of a patient includes a mandible 4. The mandible 4 includes an outer surface 6 that largely defines the appearance of the chin of the patient. Damage or deformities in the mandible 4 can cause an unnatural or undesired appearance of the chin. An implant 20 can be provided that is configured to abuts a target area 8 of the mandible 4 such that when the implant 20 is secured to the target area 8 of the mandible 4, a natural or desired appearance of the chin can be obtained or restored. The implant 20 includes an inner surface 22 and an outer surface 24 that is opposite the inner surface 22.

The inner surface 22 is configured to correspond to the outer surface 6, for instance the target area 8, of the mandible 4. In one embodiment the inner surface 22 can define a curvature, for instance a concave curvature, that corresponds to the curvature of at least a portion of the outer surface 6 of the mandible 4, for example the mental protuberance and the symphysis. The outer surface 24 is configured to correspond to a natural or desired shape of the target area 8. For example, the outer surface 24 of the implant 20 can define a curvature, for instance a convex curvature, which corresponds to a natural or undamaged outer surface 6 of the mandible 4.

The implant 20 can be made of any biocompatible material, such as high density polyethylene or ultra-high weight polyethylene. The implant can be pre-molded such that the inner surface 22 and the outer surface 24 define curvatures that correspond to the boney structure that the implant is going to be secured to as described in detail above. The implant 20 is configured to have a size, shape, and contouring that is compatible with a variety of mandibles 4 of different sizes and shapes. However, the implant 20 can also be customized, for instance the size, shape, or contouring of the implant can be adjusted to fit the specific configuration of an individual mandible 4. In one embodiment the implant 20 can be bent, shaved, or shaped to fit the specific configuration of an individual mandible 4.

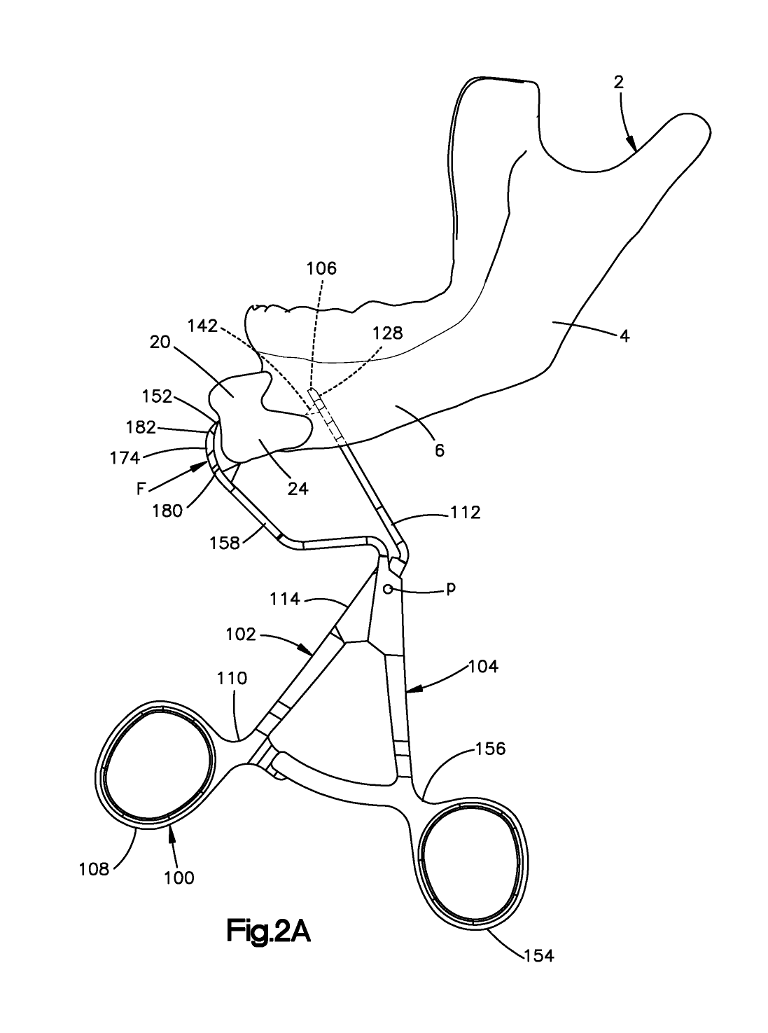

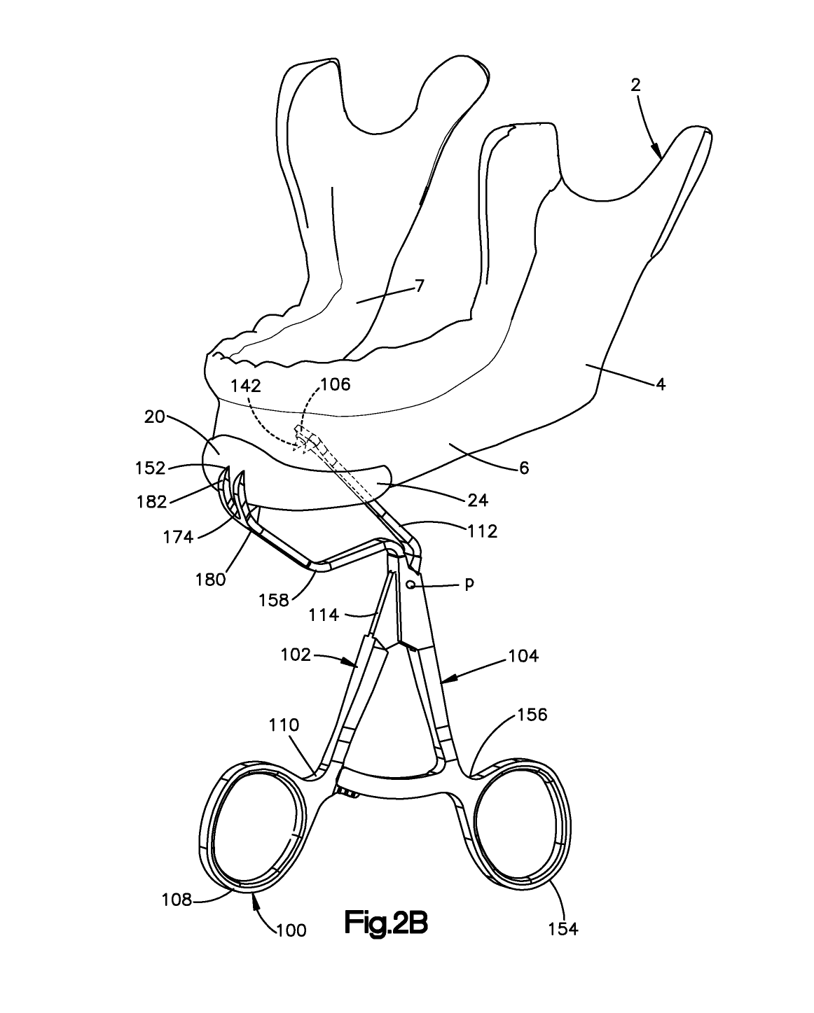

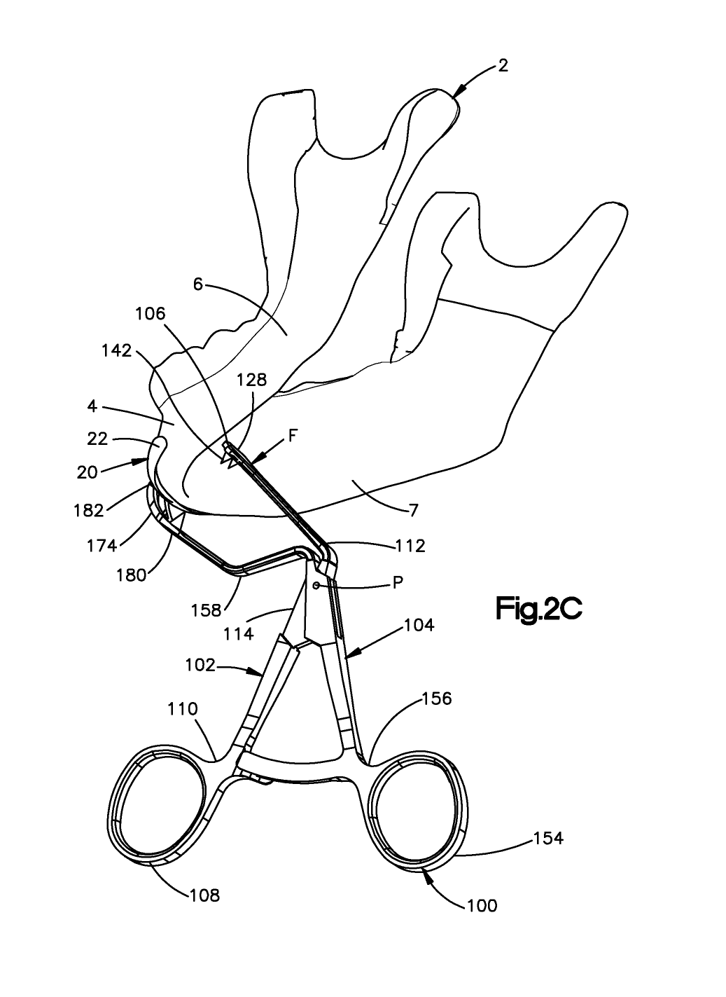

Referring to FIGS. 2A to 2C, once the implant 20 is positioned adjacent the target area 8 of the mandible 4, a surgical instrument 100 can be used to hold the implant 20 in a desired position such that the implant 20 does not move significantly during fastening of the implant 20 to the mandible 4. In one embodiment, fastening the implant 20 to the mandible 4 includes predrilling a number of holes through the inner and outer surfaces 22 and 24 of the implant 20 and at least partially through the mandible 4. Fasteners, such as bone screws, can then be inserted into the predrilled holes to either permanently or temporarily secure the implant 20 to the target area 8 of the mandible. In one embodiment the number of predrilled holes and fasteners is between 1 and 8. In another embodiment the number of predrilled holes and fasteners is between 2 and 4.

The instrument 100 includes a first arm 102 and a second arm 104 that is movably coupled to the first arm. For instance, the instrument 100 further defines a pivot axis P, such that one or both of the first and second arms 102 and 104 is pivotable with respect to the other of the first and second arms 102 and 104 about the pivot axis P. The first arm 102 can define a first proximal portion 110 that is disposed proximal with respect to the pivot axis P, a first distal portion 112 that is disposed distal with respect to the pivot axis P, and a first body portion 114 that extends between the first proximal portion 110 and the first distal portion 112, and further extends across the pivot axis P. Thus, the first distal portion 112 extends distally with respect to the first proximal portion 110 and the first body portion 114. Similarly, the second arm 104 can define a second proximal portion 156 that is disposed proximal with respect to the pivot axis P, a second distal portion 158 that is disposed distal with respect to the pivot axis P, and a second body portion 160 that extends between the second proximal portion 156 and the second distal portion 158, and further extends across the pivot axis P. Thus, the second distal portion 158 extends distally with respect to the second proximal portion 156 and the second body portion 160.

As will be described in more detail below, the instrument 100 includes a grip member 142, which can be at least partially defined by a first set of at least one protrusion 144, and the instrument 100 can further include an implant retention member 173, which can be at least partially defined by a second set of at least one protrusion 184. For instance, the first arm 102 can include the first set of at least one protrusion 144 that extends from the first distal portion 112, and the second arm 104 can include the second set of at least one protrusion 184 that extend from the second distal portion 158. The first set of at least one protrusion 144 is configured to grip, for instance frictionally grip, the mandible 4, for instance at the inner surface 7 of the mandible 4. The second set of at least one protrusion 184 is configured to abut and grip, for instance pierce, the implant 20 when the instrument 100 retains the implant 20 against the mandible 4. The second arm 104, for instance at least one or both of the second distal portion 158 and the second set of at least one protrusion 184, can further define a cradle 180 that is configured to abut a length of the implant 20 when the instrument 100 retains the implant 20 against the mandible 4.

The first arm 102 includes a first proximal portion 110, a first distal portion 112, and a first body portion 114 that extends from the first proximal portion 110 to the first distal portion 112. The first distal portion 112 is configured to engage, for instance frictionally grip an inner surface 7 of the mandible 4. The inner surface 7 of the mandible 4 can include, for example, a posterior surface of the symphysis. The inner surface 7 can be opposite the outer surface 6 of the mandible 4 which can include an anterior surface of the symphysis. In one embodiment the first distal portion 112 includes at least one grip member 142 that is configured abut the inner surface 7 of the mandible 4 and frictionally grip the mandible 4 such that translational movement or rotation of the mandible 4 relative to the first arm 102 is limited in all directions and degrees of freedom.

The second arm 104 includes a second proximal portion 156, a second distal portion 158 and a second body portion 160 that extends from the second proximal portion 156 to the second distal portion 158. The implant retention member 173 can extend from the second distal portion 158, and is configured to bias the implant 20, for instance at least a portion of the inner surface 22, against the mandible 4. For instance, the implant retention member 173 can include the cradle 180 that is configured to abut the outer surface 24 so as to bias the implant 20, for instance at least a portion of the inner surface 22, against the mandible 4, and can further include a piercing portion, which can be at least partially defined by the second set of at least one protrusion 184, that is configured to pierce the outer surface 24 of the implant 20, so as to bias the implant 20, for instance at least a portion of the inner surface 22, against the mandible 4. Thus, the implant retention member 173 can include a first member, such as the cradle 180, that is disposed at a first location of the second arm 104 and is configured to abut the implant 20 without piercing the implant 20, and a second member, such as the piercing portion 182 that is configured to pierce the implant 20, the piercing portion 182 disposed at a second location of the second arm 104 that is spaced from the first location of the second arm 104. In accordance with one embodiment, a force can be applied to the instrument 100 that causes the second distal portion 158 of the second arm 104 can be moved toward the first distal portion 112 of the first arm 102 about the pivot axis P such that the piercing portion 182 pieces the implant 20 and causes the cradle 180 to abut the implant 20. Otherwise stated, the force is sufficient to bring the cradle 180 into abutment with the implant 20, which can thus cause the piercing portion 182 to pierce the implant 20.

In accordance with the illustrated embodiment, the second distal portion 158 includes a second support arm 172 that extends out from the second body portion 160, and a second tip 174 that extends out from the second support arm 172. The second distal portion 158, for instance at the second tip 174, at least partially defines a cradle 180. It should be appreciated that the cradle 180 and the piercing portion 182 can collectively define one or more points of contact with the outer surface 24 of the implant 20. The cradle 180 is configured to abut the outer surface 24 at one or more points of contact without piercing the outer surface 24 of the implant 20. The piercing portion 182 is configured to pierce or bite into the outer surface 24 of the implant 20 (but not puncture all the way through to the inner surface 22 of the implant 20).

When the implant 20 is in the desired position abutting the mandible 4, the surgical instrument 100 can be positioned such that the mandible 4 and the implant 20 are disposed between the first arm 102 and the second arm 104 such that the first arm 102 is adjacent the inner surface 7 of the mandible 4 and the second arm 104 is adjacent the outer surface 24 of the implant. The first and second arms 102 and 104 can then be rotated in a first direction with respect to each other about the pivot axis P, such that the first distal portion 112 and the second distal portion 158 move toward each other until the grip member 142 contacts the inner surface 7 of the mandible 4, the cradle 180 abuts the outer surface 24 of the implant 20, and the piercing portion 182 pierces the outer surface 24 of the implant 20. As the first and second arms 102 and 104 are rotated further in the first direction the grip member 142 of the first tip 128 applies a force F to the inner surface 7 of the mandible 4 and the cradle 180 and piercing portion 182 of the second tip 174 apply a force F to the outer surface 24 of the implant 20 such that the implant 20 and the mandible 4 are secured in a desired position relative to each other and do not move relative to each other.

The grip member 142, the cradle 180, and the piercing potion 182 are each configured such that the force F is applied to the mandible 4 and the implant 20 such that at least a portion of the inner surface 22 of the implant 20 lays substantially flush against the outer surface 6 of the mandible 4. The force F used to achieve a flush fitting between the implant 20 and the mandible 4 can vary depending on the material and shape of the implant 20, the area of the mandible 4 that the implant 20 is being attached to, as well as numerous other factors as will be understood by a person of skill in the art. For example if the implant 20 is being secured against a midline of the mandible 4, more force F may be require for a flush fit than at a region of the mandible 4 spaced laterally away from the midline of the mandible 4.

In one embodiment the force F can vary between about 1 Newton (“1 N”) and about 150 N. In another embodiment the force F can vary between about 5 N and about 10 N. Application of the force F to the implant 20 and the mandible 4 as described above may result in the implant 20 laying flush against the outer surface 6 of the mandible 4 such that there are no gaps between the inner surface 22 of the implant 20 and the outer surface 6 of the mandible. Once the implant 20 has been positioned against the mandible 4 as described above, the implant 20 can be permanently or temporarily secured to the mandible 4 by inserting fasteners, such as bone screws, through the implant 20 and into the mandible 4.

As shown in the illustrated embodiment, the second tip 174 includes both the cradle 180 and the piercing portion 182 as described above. The cradle 180 and the piercing portion 182 collectively define the one or more points of contact between the second tip 174 and the implant 20. In another embodiment the second tip 174 can include only one of either the cradle 180 or the piercing portion 182 that defines all of the one or more points of contact between the second tip 174 and the implant 20. In another embodiment the second tip 174 includes both the cradle 180 and the piercing portion 182 as described above but only one of the cradle 180 and the piercing portion 182 defines all of the one or more points of contact between the implant 20 and the second tip 174.

Although the instrument 100 is described above in use for securing an implant 20 to a mandible 4, it should be understood that the instrument 100 is not so limited. In one embodiment the instrument 100 can be used to secure and compress an implant to another boney structure in a human body. In another embodiment the instrument 100 can be used to secure a first surface relative to a second surface such that the first and second surfaces are compressed towards one another to minimize or eliminate any gaps between the first and second surfaces.

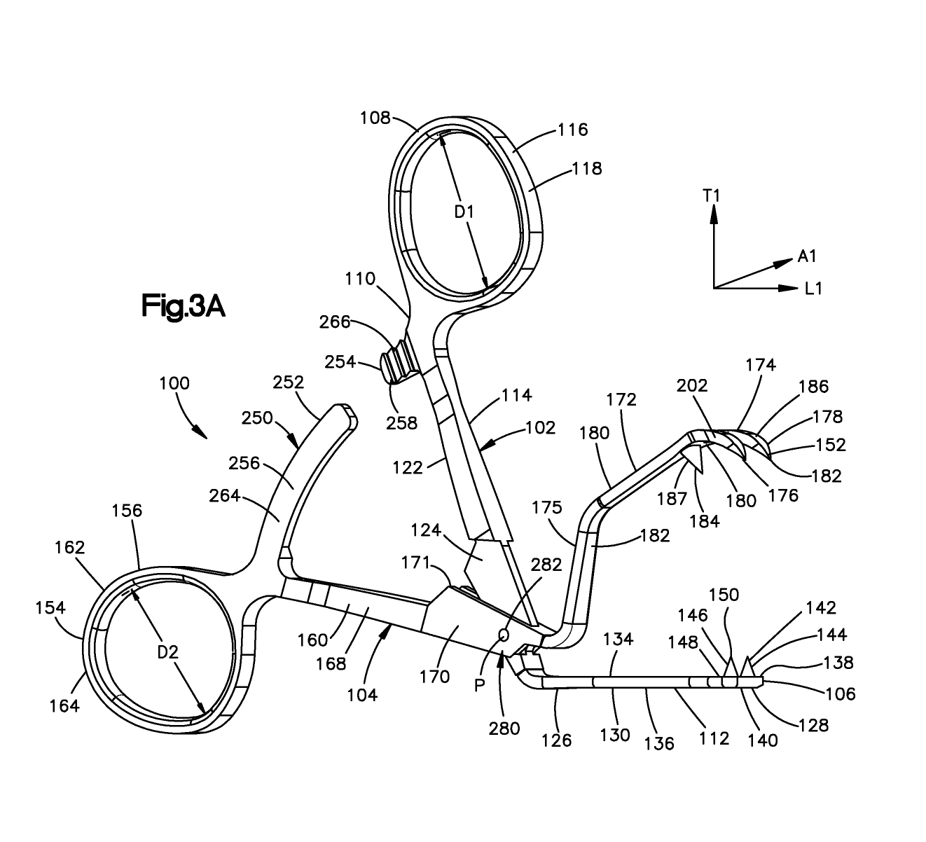

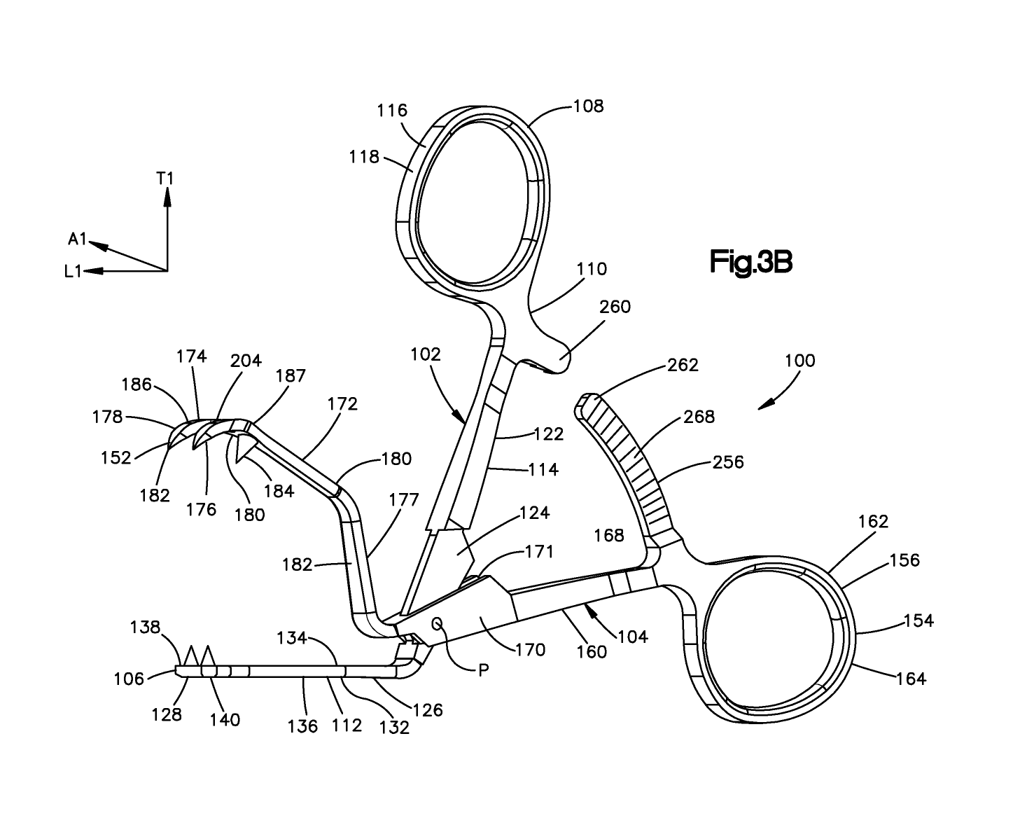

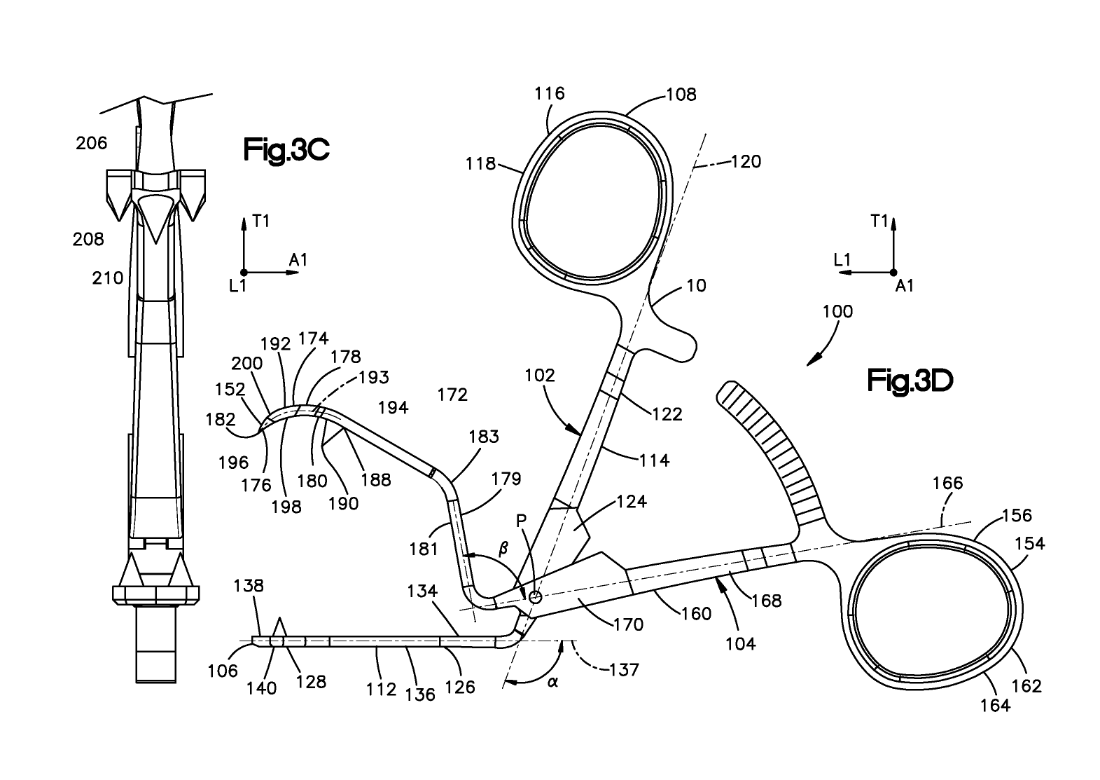

Referring to FIGS. 2A to 3D, and as described above, the surgical instrument 100 includes the first arm 102 and the second arm 104 that is movably coupled, for instance rotatably coupled about a pivot axis P, to the first arm 102. The first arm 102 includes the first proximal portion 110, the first distal portion 112 and the first body portion 114 that extends from the first proximal portion 110 to the first distal portion 112. The first proximal portion 110 can include a first handle 116 that can be constructed as desired. In accordance with the illustrated embodiment, the first handle 116 can include a first ring member 118 that defines a first inner dimension D1. The first ring member 118 is configured to receive a force F to manipulate the position of the first arm 102 relative to the second arm 104. In one embodiment the first inner dimension D1 is configured to receive a human finger that applies the force F to manipulate the position of the first arm 102 relative to the second arm 104.

The first body portion 114 defines a first central axis 120, the first body portion 114 being elongate along the first central axis 120. The first body portion 114 includes a first shaft member 122 and a first pivot portion 124. The first shaft member 122 extends along the first central axis 120 from the first proximal portion 110 to the first pivot portion 124. The first pivot portion 124 extends along the first central axis 120 from the first shaft member 122 to the first distal portion 112.

A first three dimensional coordinate system is provided in reference to the first distal portion 112 of the first arm 102. As shown in the illustrated embodiment, the first distal portion 112 includes a first support arm 126 and a first tip 128 that extends distally from the first support arm 126. The first support arm 126 extends distally from the first pivot portion 124 to the first tip 128, which can extend from the first support arm 126 in a first longitudinal direction L1. The first support arm 126 can be elongate in the first longitudinal direction L1 from the first pivot portion 124 to the first tip 128.

The first support arm 126 includes first and second opposed side walls 130 and 132 that each extends from the first pivot portion 124 to the first tip 128 in the first longitudinal direction L1. The first and second opposed side walls 130 and 132 are spaced apart in a first lateral direction A1 that is perpendicular to the first longitudinal direction L1. The first support arm 126 further includes a first top surface 134 and an opposed first bottom surface 136 that each extends from the first pivot portion 124 to the first tip 128 in the first longitudinal direction L1. The first top surface 134 and the first bottom surface 136 are spaced apart in a first transverse direction T1 that is perpendicular to both the first longitudinal direction L1 and the first lateral direction A1.

The first support arm 126 defines a second central axis 137 that extends through the first support arm 126 from the first pivot portion 124 in the first longitudinal direction L1. As shown in the illustrated embodiment, the second central axis 137 can be angularly offset with respect to the first central axis 120 of the first body portion 114 so as to define an angle α that is configured such that when the first arm 102 is engaged with a boney structure, such as a lingal side of a mandible, the first proximal portion 110 is positioned so as not to interfere with any other nearby boney structure such as a sternum or ribcage.

In one embodiment the angle α can between 0 degrees and 180 degrees. In another embodiment the angle α can be between about 90 degrees and about 145 degrees. In another embodiment the first and second central axes 120 and 137 can be aligned such that the angle α is 0 degrees. The angle α is configured such that when the first arm 102 is engaged with a first surface, such as the inner surface 7 of the mandible 4, and the second arm 104 is engaged with a second surface, such as the outer surface 22 of the implant 20, the handles are positioned so as not to interfere with any structure attached to the first or second surfaces, such as a sternum.

The first tip 128, as shown, includes a first inner surface 138 and an opposed first outer surface 140. The first inner surface 138 carries the grip member 142 that can be carried by, for instance extend out from, the first tip 128, for instance the first inner surface 138 of the first tip 128, along a direction that is substantially perpendicular to the first inner surface 138. The grip member 142 is configured to grip, a first surface, such as the inner surface 7 of the mandible 4 during fixation of the implant 20 to the target area 8 of the mandible 4.

As shown in the illustrated embodiment, the first arm 102, and in particular the first distal portion 112, can include a grip member 142 that is configured to frictionally grip a first surface, which can be defined by the mandible 4, once the first tip 128 has been moved into contact with the first surface. In accordance with the illustrated embodiment, the grip member 142 can include a first set of one or more protrusions 144 (referred to hereafter as “first protrusions 144”). The first protrusions 144 can include a single first protrusion 144 or a plurality (at least two) of first protrusions 144 as described in detail below. In one embodiment, the first protrusions 144 can be in the form of one or more first spikes 146 each having a relatively wide base 148 and tapering to an apex 150. The first protrusions 144 can taper along a direction that extends substantially perpendicularly out from the first inner surface 138. The first spikes 146 each extend out from the base 148, which is positioned adjacent the first inner surface 138, to the apex 150 in the first transverse direction Ti. Each of the first spikes 146 defines a height measured from the base 148 to the apex 150 in the first transverse direction T1. In one embodiment the height of each of the first protrusions 144 is constant. In another embodiment the height of each of the first protrusions 144 is varied.

A first tip 128 that supports multiple first protrusions 144, such as two or more, may help reduce rotation of the grip member 142 relative to the first surface, which may be desirable. However it will be appreciated by those skilled in the art that the grip member 142 can include a single first protrusion 144. The first protrusions 144 are shown as conical shaped first spikes 146. It will be appreciated by those skilled in the art that first protrusion 144 can be any shape configured to frictionally grip the first surface when the first tip 128 is in contact with the first surface. For example, the first protrusions 144 could be in the form of any one of or combination of a spike with a flattened top, a blade, a textured surface, etc.

As shown in the illustrated embodiment, the first protrusions 144 (two spikes 146) are arranged such that the spikes 146 are longitudinally aligned in the first longitudinal direction L1, transversely aligned in the first transverse direction T1, and laterally spaced relative to each other on the first inner surface 138 in the first lateral direction A1. It will be appreciated by those skilled in the art that the first protrusions 144 can be positioned in alternate arrangements such as any combination of one or more of longitudinally spaced, laterally spaced, and transversely spaced. In one embodiment the first protrusions 144 can include three protrusions arranged in a triangular orientation.

As described above, and with continuing reference to FIGS. 2A-4D, the second arm 104 includes the second proximal portion 156, the second distal portion 158 and the second body portion 160 that extends distally from the second proximal portion 156 to the second distal portion 158. The second proximal portion 156 can include a second handle 162 that can be configured as desired. For instance, the second handle 162 can include a second ring member 164 that defines a second inner dimension D2. The second ring member 164 is configured to receive a force F to manipulate the position of the second arm 104 relative to the first arm 102. In one embodiment the second inner dimension D2 is configured to receive a human finger that applies the force F to manipulate the position of the second arm 104 relative to the first arm 102.

The second body portion 160 defines a third central axis 166, the second body portion 160 being elongate along the third central axis 166. The second body portion 160 includes a second shaft member 168 and a second pivot portion 170. The second shaft member 168 extends along the third central axis 166 from the second proximal portion 156 to the second pivot portion 170. The second pivot portion 170 extends along the third central axis 166 from the second shaft member 168 to the second distal portion 158.

A second three dimensional coordinate system is provided in reference to the second distal portion 158 of the second arm 104. As shown in the illustrated embodiment, the second distal portion 158 includes a second support arm 172 connected to the second pivot portion 170, and a second tip 174 that extends out from the second support arm 172. As shown in the illustrated embodiment, as the second support arm 172 extends from the second pivot portion 170 to the second tip 174, at least partially in a second longitudinal direction L2. The second support arm 172 includes third and fourth opposed side walls 175 and 177 that each extends from the second pivot portion 170 to the second tip 174 in the second longitudinal direction L2. The second support arm 172 the third side wall 175 is spaced apart from the fourth side wall 177 in a second lateral direction A2 that is perpendicular to the second longitudinal direction L2. The second support arm 172 further includes a second top surface 179 and an opposed second bottom surface 181 that each extends from the second pivot portion 170 to the second tip 174 in the second longitudinal direction L2. The second top surface 179 is spaced apart from the second bottom surface 181 in a second transverse direction T2 that is perpendicular to both the second longitudinal direction L2 and the second lateral direction A2.

The second support arm 172 defines a fourth central axis 183 that extends through the second support arm 172 from the second pivot portion 170 at least partially in the second longitudinal direction L2. As shown in the illustrated embodiment, the fourth central axis 183 can be angularly offset with respect to the third central axis 166 of the second body portion 160 so as to define an angle β that is configured to create a separation distance between the first and second tips 128 and 174. In one embodiment the angle β can between 0 degrees and 180 degrees. In another embodiment the angle β can be between about 45 degrees and about 90 degrees.

The second tip 174, as shown, includes a second inner surface 176 that faces the first inner surface 138 and a second outer surface 178 that is opposite the second inner surface 176. The second inner surface 176 faces and is configured to engage, for instance frictionally grip, a second surface, such as the outer surface 24 of the implant 20 when the instrument 100 is fixed to the mandible 4. The second outer surface 178 can be curved, and can be substantially devoid of sharp edges so to avoid interference with (and thus irritation of) soft tissue that the second outer surface 178 may come into contact with. The second distal portion 158 can include the implant retention member 173 which can be carried by the second tip 174. Thus, the second distal portion 158 can include the cradle 180 and the piercing portion 182 that can each be carried by the second tip 174. The cradle 180 and the piercing portion 182 are each configured to independently engage the outer surface 24 of the implant 20, so as to bias or compress the inner surface 22 of the implant 20 is towards the outer surface 6 of the mandible 4. Collectively, the cradle 180 and the piercing portion 182 define one or more points of contact that engage with the second surface such that upon rotation of the first and second handle portions 110 and 156 towards each other about the pivot axis P, the second surface is compressed towards the first surface.

As shown in the illustrated embodiment, the cradle 180 can include at least one protrusion, such as a second spike 187, of the second set of one or more protrusions 184 (referred to hereafter as “second protrusion 184”). The piercing portion 182 can include at least one other protrusion of the second set of one or more protrusions. For instance, the protrusions of the piercing portion 182 can be constructed as first and second teeth 186 that are configured to pierce the outer surface 24 of the implant 20. Thus, the second set of one or more protrusions can include at least one second spike 187 and at least one, such as a pair of, teeth 186. In accordance with the illustrated embodiment, the second tip 174 can be forked so as to define the first and second teeth 186. The second spike 187 is configured to abut or frictionally grip the implant 20 once the second tip 174 has been moved into contact with the implant 20. In one embodiment, the second spike 187 can include a second base 188 that is positioned adjacent the second inner surface 176, and can be tapered from the second base 188 in the second transverse direction T2 to a second apex 190.

The second spike 187 can define a second height measured from the second base 188 to the second apex 190 in the second transverse direction T2. In one embodiment the height of the second protrusions 184 is different than, for instance greater than or less than, the first height of at least one of the first protrusions 144. In another embodiment the second height of the second protrusions 184 is the same as the first height of at least one of the first protrusions 144.

The teeth 186 each include a tooth body 192 that includes a tooth base 194 and a tooth apex 196 that at least partially defines the piercing portion 182. Each tooth body 192 can include a tooth inner end 198, a tooth outer end 200 that is opposite the tooth inner end 198, and first and second tooth sides 202 and 204 that each extend from the tooth inner end 198 to the tooth outer end 200. As shown in the illustrated embodiment, the tooth body 192 is curved such that the teeth 186 extend from the tooth base 192 to the tooth apex 196 in both the second longitudinal direction L2 and in the second transverse direction T2. The tooth body 192 includes a tooth central axis 193 that extends from the tooth base 194 to the tooth apex 196. As shown the tooth central axis 193 can be curved. The tooth body 192 defines a tooth length measured from the tooth base 194 to the tooth apex 196 along the tooth central axis 193. The tooth body 192 defines a tooth width measured between the first and second tooth side walls 202 and 204 in the second lateral direction A2 or measured in a direction substantially perpendicular to the tooth central axis 193. The tooth body 192 further defines a tooth height measured between the tooth inner end 198 and the tooth outer end 200 in the second transverse direction T1 that is substantially perpendicular to both the second lateral direction A2 and the tooth central axis 193.

The tooth body 192 can include a first tooth portion 206 and a second tooth portion 208, wherein the second tooth portion 208 is located distally of the first tooth portion 206. The first tooth portion 206 can define a constant cross-sectional area such that the tooth width and the tooth height are constant along first portion 206. The second tooth portion 208 can define a varying cross-sectional area such that the second tooth portion 208 is tapered as the tooth body 192 extends distally from the tooth base 194 to the tooth apex 196. The second tooth portion 208 can be tapered such that either the tooth width, the tooth height, or both the tooth width and the tooth height decrease as the tooth body 192 extends from the tooth base 194 to the tooth apex 196. The second tooth apex 196 defines a leading edge 210. In one embodiment the leading edge 210 is in the form of a point, for example if the tooth width and the tooth height taper down towards the tooth apex 196 such that the tooth inner end 198, the tooth outer end 200, the first tooth side wall 204, and the second tooth side wall 206 all meet at the tooth apex 196. In another embodiment, the leading edge 210 is in the form of a line, for example if either the tooth width or the tooth height tapers down towards the tooth apex 196 such that either the tooth inner end 198 and the tooth outer end 200; or the first tooth side wall 204 and the second tooth side wall 206 meet at the tooth apex 196.

As shown in the illustrated embodiment, the cradle 180 can be defined by the tooth inner end 198 of the teeth 186 alone or in combination with the second spike 187. The cradle 180 can be configured to cradle or abut the outer surface 24 of the implant 20 at a location defined by and between the second spike 187, for instance the distal-facing surface of the second spike 187, and the apices 196 of the teeth 186 such that one or more points of contact are defined between the implant 20 and the cradle 180. In one embodiment the one or more points of contact include the second spike 187 and the tooth inner end 198 of at least one of the one or more teeth 186. The tooth inner end 198 is configured to correspond in shape to the outer surface 24 of the implant 20 such that a portion of the tooth inner end 198 cradles or abuts the outer surface 24 of the implant 20 to create one or more points of contact between the second tip 174 and the outer surface 24 of the implant 20. In accordance with the illustrated embodiment, the abutment between the cradle 180 and the implant 20 can span at least 10%, such as 25%, for instance at least 50% of the distance between the second spike 187 and the apices 196 of the teeth 186.

The piercing portion 182 can be configured to pierce or bite into the implant 20 such that the piercing portion passes through the outer surface 24 of the implant 20 but does not puncture all the way through the implant 20 such that the piercing portion 182 exits the inner surface 22 of the implant 20. The piercing portion 182 includes the second tooth portion 208 of each of the one or more teeth 186, which extends distally out from the first tooth portion 206.

In one embodiment the piercing portion 182 includes two teeth 186 that are offset with respect to each other in the second lateral direction A2 and aligned with respect to each other in both the second longitudinal direction L2 and the second transverse direction T2. In another embodiment the two teeth 186 can be aligned with respect to one another in the second lateral direction A2 and offset in at least one of the second longitudinal direction L2 and the second transverse direction T2.

As shown the two teeth 186 can be identical such that the two teeth have the same tooth length, tooth height, and tooth width. In another embodiment the two teeth 186 can be different such that the two teeth 186 have at least one of a different tooth length, tooth height, and tooth width.

As shown in the illustrated embodiment, the one or more teeth 186 includes two teeth 186. In another embodiment the one or more teeth 186 includes a single tooth 186 that is configured to pierce or bite into the implant 20. In another embodiment the one or more teeth 186 can include multiple teeth 186 such as 3 or more teeth 186 arranged in any of the orientations described in detail above.

In one embodiment the first protrusions 144 can each be integral (or monolithic) with the first inner surface 138, respectively such that the first protrusions 144 and the first inner surface 138 are formed of a single piece of material. In another embodiment the second protrusions 184 can each be integral (or monolithic) with the second inner surface 176, respectively such that the first protrusions 144 and the first inner surface 138 are formed of a single piece of material. In another embodiment, the one or more first protrusions 144 are removable from the first inner surface 138 such that the one or more first protrusions 144 can be switched out, for example spikes 146 could be removed and replaced with a textured surface (not shown).

The instrument 100 can include a locking mechanism 250 configured to retain the first arm 102 to the second arm 104 in a relative orientation about the pivot axis P. In one embodiment the locking mechanism 250 is included in the first and second handle portions 110 and 156. In another embodiment the locking mechanism 250 can be included in the first and second body portions 114 and 160. In another embodiment the locking mechanism 250 can be included in the first and second distal portion 112 and 158. The locking mechanism 250 can be configured to retain the first arm 102 in a relative orientation to the second arm 104 about the pivot axis P such that no hands are required on the instrument to maintain the relative orientation.

As shown in the illustrated embodiment the locking mechanism can include a ratchet system 252. The ratchet system 252 includes a first ratchet arm 254 and a second ratchet arm 256. The first ratchet arm 254 includes a first ratchet arm inner surface 258 and a first ratchet arm outer surface 260. Similarly, the second ratchet arm 256 includes a second ratchet arm inner surface 262 and a second ratchet arm outer surface 264. The first and second ratchet arm inner surface 258 and 262 include corresponding sets of teeth 266 and 268 respectively.

The teeth 266 and 268 are configured such that as the first and second arms 102 and 104 are rotated about the pivot axis P in the first direction, such that the first and second tips 128 and 174 move closer to one another the teeth 266 and 268 slide over each other. However, rotation in the second direction, opposite the first direction, is blocked by the teeth 266 and 268 interfering with each other and not sliding over each other. Thus, when the first and second arms 102 and 104 have been rotated about the pivot axis P in the first direction such that a compressive force F is applied to the implant 20 and the mandible 4, the first and second handle portions 110 and 156 can be released and the first and second arms 102 and 104 will remain in their current orientation and continue to apply the compressive force F to the implant 20 and mandible 4.

The instrument 100 can further include a hinge assembly 280 that includes the first and second body portions 114 and 160 and the pivot axis P. As shown in the illustrated embodiment the first pivot portion 124 of the first body portion 114 is pivotally coupled to the second pivot portion 170 of the second body portion 160 at the pivot axis P such that the first and second body portions 114 and 160 (and thus the first and second arms 102 and 104) can rotate about the pivot axis P relative to each other. In one embodiment the first pivot portion 124 passes through a recess 171 or opening in the second pivot portion 170. The first and second pivot portions 124 and 170 can be joined by a pin 282 that passes at least partially through the first and second pivot portions 124 and 170. The pin 282 can be laser welded or staked to at least one of the first and second pivot portions 124 and 170 to prevent disassembly of the first and second arms 102 and 104.

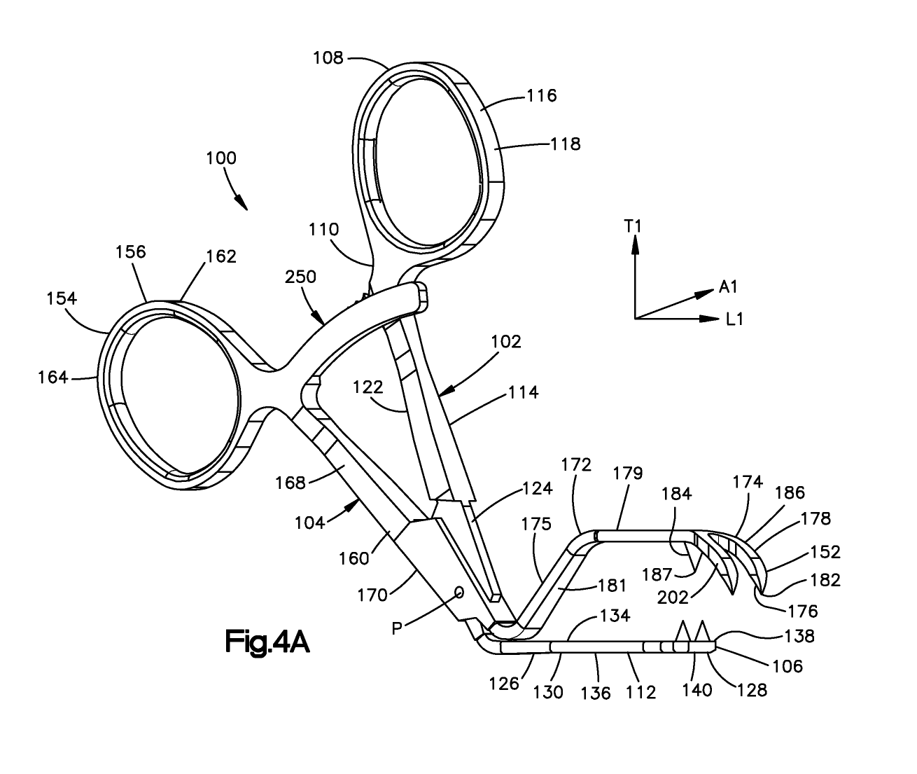

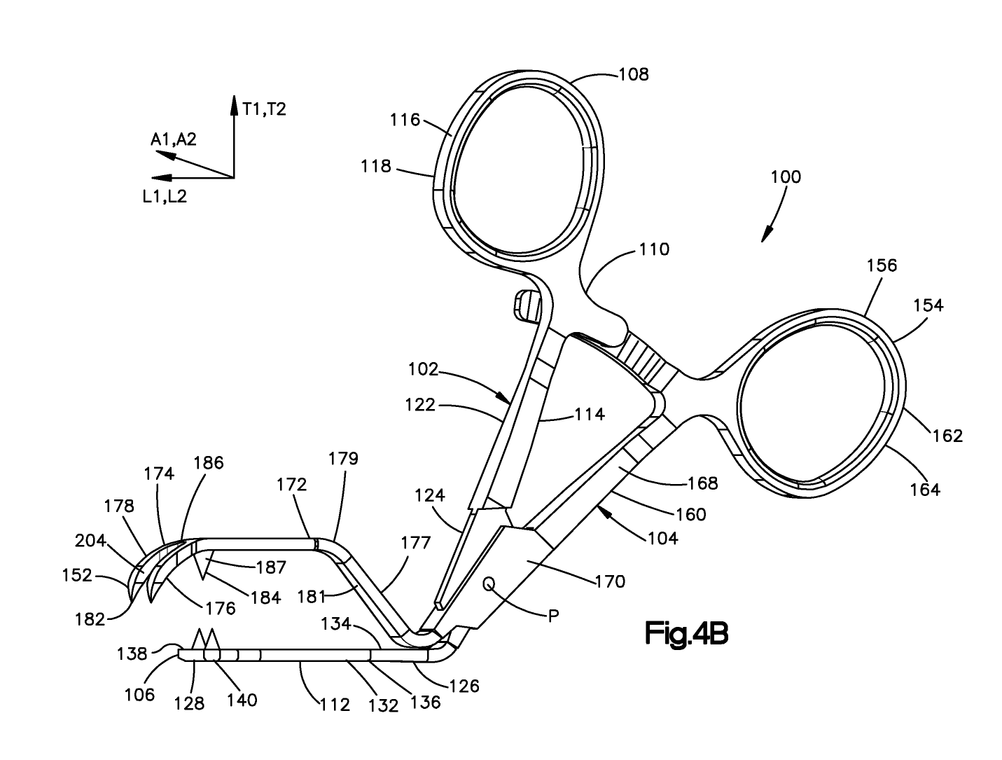

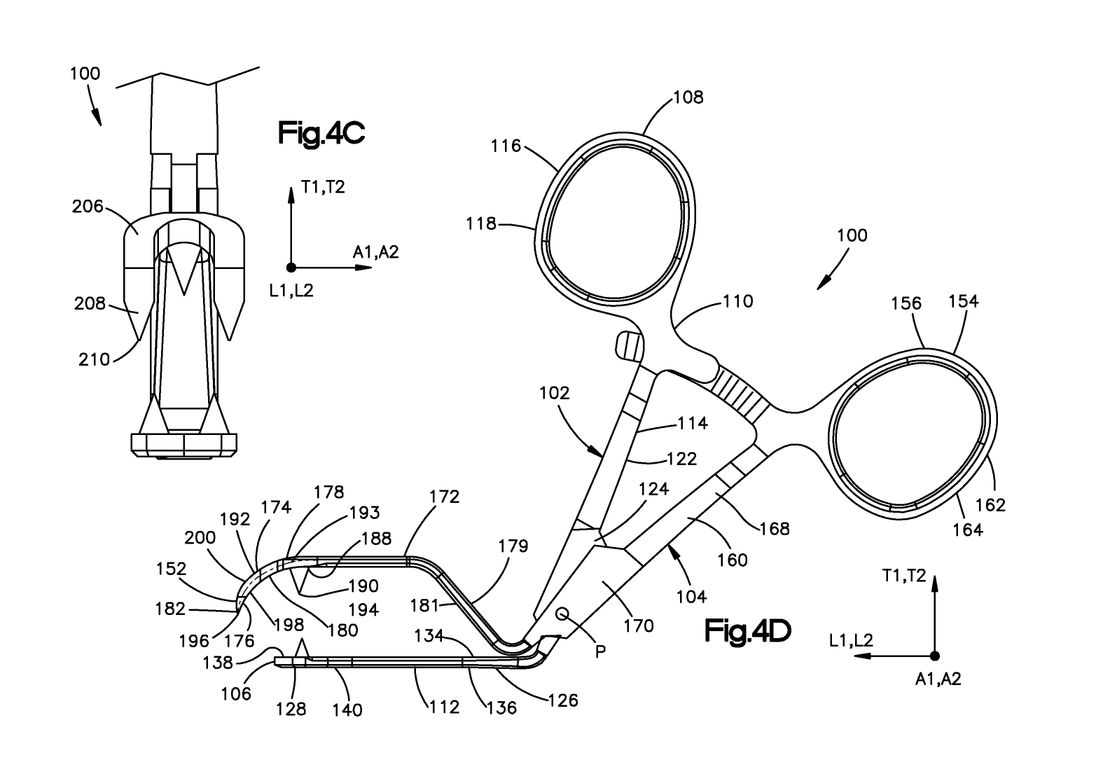

Referring to FIGS. 3A to 4D, the instrument 100 includes a first configuration, for example an open configuration (as shown in FIGS. 3A to 3D) and a second configuration, for example a closed configuration (as shown in FIGS. 4A to 4D). In the first configuration the first inner surface 138 of the first tip 128 and the second inner surface 176 of the second tip 174 are separated by a distance D. The distance D, in the first configuration is configured such that an implant 20 that is abutting a mandible 4 will fit between the first inner surface 138 of the first tip 128 and the second inner surface 176 of the second tip 174. In the second configuration the distance D separating the first and second inner surfaces 138 and 176 is reduced relative to the distance D when the instrument 100 is in the first configuration. In the closed configuration the distance D is configured such that the first and second inner surfaces 138 and 176 can engage and apply a compressive force F to two opposed surfaces, such as the outer surface 24 of the implant 20 and the inner surface 7 of the mandible 4.

In one embodiment the pivot axis P is parallel to the first and second lateral directions A1 and A2. As the first arm 102 rotates relative to the second arm 104 about the pivot axis P in the first direction such the instrument 100 transitions from the first configuration to the second configuration. As the instrument 100 transitions from the first configuration to the second configuration the relative positions of the grip member 142 and the cradle and piercing portions 180 and 182 may change. For example, in one embodiment when the instrument 100 is in the first configuration the grip member 142 of the first tip 128 is positioned farther from the pivot axis P than the cradle 180 and the piercing portion 182 (as measured along the first longitudinal direction L1). As the second arm 104 is rotated relative to the first arm 102 in the first direction transitioning the instrument 100 to the second configuration, at least a portion of the cradle 180 or the piercing portion 182 can be positioned farther from the pivot axis P than the grip member 142 (as measured along the first longitudinal direction L1).

While the foregoing description and drawings represent the preferred embodiments of the present invention, it will be understood that various additions, modifications, combinations and/or substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed embodiments are therefore to be considered in all respects as illustrative and not restrictive, the scope of the invention being indicated by the appended claims, and not limited to the foregoing description.

What is claimed:

1. A surgical instrument configured to retain an implant against a mandible, the surgical instrument comprising:
a first arm including a first proximal portion, a first distal portion, and a first body portion that extends distally from the first proximal portion to the first distal portion, the first distal portion including at least one grip member configured to grip the mandible; and
a second arm that is pivotally coupled to the first arm about a pivot axis, the second arm including a second proximal portion, a second distal portion, and a second body portion that extends distally from the second proximal portion to the second distal portion, the second distal portion carrying an implant retention member that is configured to abut the implant at a first location of the second arm, and further carrying a piercing portion that is configured to pierce the implant, the piercing portion disposed at a second location of the second arm that is spaced from the first location of the second arm.

2. The instrument of claim 1, wherein the first distal portion includes a first inner surface that faces the second arm, and the grip member includes at least one first protrusion that extends out from the first inner surface.

3. The instrument of claim 2, wherein the one or more first protrusions include two conical spikes.

4. The instrument of claim 2, wherein the at least one first protrusion tapers along a direction that extends substantially perpendicularly out from the first inner surface.

5. The instrument of claim 2, wherein the one or more first protrusions include at least one of a spike with a flattened top, a blade, and a textured surface.

6. The instrument of claim 2, wherein the grip member is configured to frictionally grip but not pierce the first surface.

7. The instrument of claim 1, wherein the implant retention member includes at least one cradle that is configured to abut the implant, and at least one piercing portion that is configured to pierce the implant.

8. The instrument of claim 7, wherein the second distal portion includes at least one spike and at least one tooth having a tooth apex, the cradle extends from the spike to the apex, and the apex that at least partially defines the piercing portion.

9. The instrument of claim 8, wherein the spike is substantially conical.

10. The instrument of claim 8, wherein the at least one tooth includes a pair of teeth that are spaced apart from each other.

11. The instrument of claim 8, wherein the tooth apex is tapered so as to define a leading edge configured to pierce the second surface.

12. The instrument of claim 11, wherein the tooth apex is tapered with respect to two directions such that the leading edge is in the form of a point.

13. The instrument of claim 11, wherein the tooth apex is tapered with respect to one direction such that the leading edge is in the form of a line.

14. The instrument of claim 1, wherein rotation of the first arm relative to the second arm about the pivot axis in a first direction causes the first distal portion to move toward the second distal portion.

15. The instrument of claim 14, wherein rotation of the first arm relative to the second arm about the pivot axis in a second direction causes the first distal portion to move away from the second distal portion.

16. The instrument of claim 15, further comprising a locking mechanism that is configured to prevent rotation of the first arm relative to the second arm about the pivot axis in the second direction.

17. The instrument of claim 16, wherein the locking mechanism includes first and second ratchet arms attached to the first and second arms, respectively, the first and second ratchet arms each including corresponding teeth that cam over each other when the first arm is rotated with respect to the second arm in the first direction, and the corresponding teeth interfere with each other when the first arm is rotated with respect to the second arm in the second direction.

18. The instrument of claim 1, wherein the first body portion is elongate along a first central axis, and the first tip portion is elongate along a second central axis, the first central axis and the second central axis intersect to define an angle.

19. The instrument of claim 18, wherein the first central axis and the second central axis are angularly offset such that the angle is between about 90 degrees and about 145 degrees.
